(19) United States
(12) Patent Application Publication
(10) Pub. No.: US 2005/0228370 A1
(43) Pub. Date: Oct. 13, 2005

(54) Balloon catheter designs which incorporate an antenna cooperatively situated with respect to an external balloon surface for use in treating diseased tissue of a patient

(76) Inventors: Fred Sterzer, Lawrence Township, NJ (US); Daniel D. Mawhinney, Livingston, NJ (US)

Correspondence Address: FRED STERZER;MMTC, INC., SUITE A-203, 12 ROSZEL ROAD, PRINCETON, NJ, 08540, US

(21) Appl. No.: 10/822,367

(22) Filed: Apr. 12, 2004

(51) Int. Cl. A61B 18/04

(52) U.S. Cl. 606/33; 607/101; 607/156

(57) ABSTRACT

In a first embodiment, the antenna cooperatively situated with respect to an external surface of the balloon of a balloon catheter has a spiral configuration, which renders this external antenna highly directional. This first embodiment of balloon catheter is shown inserted in the urethra of a patient in a system for irradiating his prostatic tumor and measuring the tumor's temperature with a radiometer. In a second embodiment, the antenna cooperatively situated with respect to an external surface of the balloon of a balloon catheter has a helical configuration, which renders this external antenna omnidirectional.

### BACKGROUND OF THE INVENTION

1. Field of the Invention

This invention relates to inflatable balloon catheter designs that incorporate an antenna which is used to treat diseased tissue of a patient with radiation from the antenna and, more particularly, to such balloon catheter designs in which the antenna is cooperatively situated with respect to an external balloon surface.

2. Description of the Prior Art

Known are many inflatable balloon catheter designs that incorporate an antenna which is used to treat diseased tissue of a patient with radiation from various types of antennas, but in all cases the antenna is internally situated within the balloon, In this regard, reference is made to the following prior art:

U.S. Pat. No. 5,007,437, issued to Fred Sterzer on Apr. 16, 1991, entitled “Catheters for Treating Prostate Disease” discloses applying squeezing pressure to a diseased prostate, by means of a urethral and/or rectal catheter incorporating an inflatable prostate balloon, to compress the prostate while it is being irradiated from a microwave antenna, that is internally situated within the balloon increases the therapeutic temperature to which the prostate tissue more distal to the microwave antenna can be heated without heating any non-prostate tissue beyond a maximum safe temperature, and reduces the temperature differential between the heated more distal and more proximate prostate tissue from the microwave antenna.

U.S. Pat. No. 5,992,419, issued to Sterzer et al. on Apr. 16, 1999, entitled “Method Employing a Tissue-Heating Balloon Catheter to Produce a “Biological Stent’ in an Orifice or Vessel of a Patient's Body” discloses a balloon catheter inserted in the urethra, which, catheter incorporates a microwave antenna that is internally situated within the balloon, to first temporarily widen by squeezing pressure on urethral tissue thereof applied by the inflation of the balloon and then microwave energy radiated from the antenna sufficient to form the “biological stent” is applied to the urethral tissue.

U.S. patent application Ser. No. 10/337,159, filed by Sterzer et al. on Jan. 7, 2003, entitled “Inflatable Balloon Catheter Structural Designs and Methods for Treating Diseased Tissue of a Patient” discloses various types of inflatable balloon catheter designs, each of which incorporate (1) a microwave antenna that is internally situated within the balloon, (2) an insertion needle and (3) operates as an interstitial probe, for treating sub-coetaneous diseased tissue of a patient, such as (1) deep-seated tumors and (2) varicose veins.

Further, reference is made to U.S. Pat. No. 4,190,053, issued to Fred Sterzer on Feb. 26, 1980, entitled “Apparatus and Method for Hyperthermia Treatment”, which discloses the combination of both (1) apparatus for the heating of diseased tissue of a patient with radiated microwave energy and (2) a microwave radiometer for accurately measuring the temperature of the heated diseased tissue.

### SUMMARY OF THE INVENTION

The invention is directed to an improvement in a balloon catheter incorporating an antenna suitable for use in treating diseased tissue of a patient with radiation transmitted from the antenna. In accordance with the improvement, the antenna is an external antenna that is situated outside of the balloon of the catheter in cooperative relationship with a longitudinal external surface of the balloon.

### BRIEF DESCRIPTION OF THE DRAWING

FIG. 1 shows an embodiment of a prior-art balloon catheter for treating prostate disease that incorporates an antenna situated within the interior of the catheter balloon;

FIGS. 2a, 2b and 2c show various aspects of an experimental embodiment of the present invention in which a balloon catheter incorporates an antenna situated in cooperative relationship with respect to an external balloon surface;

FIGS. 3a and 3b show, respectively, a longitudinal front view and an end view of a first preferred embodiment of a balloon catheter of the present invention in which the balloon is in a deflated state;

FIGS. 4a and 4b show, respectively, a longitudinal front view and an end view of the first preferred embodiment of the balloon catheter of the present invention in which the balloon is in an inflated state;

FIGS. 5a and 5b show, respectively, a longitudinal view and an end view of the first preferred embodiment of the balloon catheter of the present invention in which the inflated balloon is shown rotated 90° with respect to the views shown in FIGS. 4a and 4b;

FIG. 6 schematically shows the first preferred embodiment of the inflated balloon catheter of the present invention employed in a system for treating a patient's prostate malignant tumor;

FIG. 7a shows an external longitudinal view of a second preferred embodiment of an inflated balloon catheter of the present invention; and

FIG. 7b shows a longitudinal cutaway view of the second preferred embodiment of the inflated balloon catheter of the present invention shown in FIG. 7a, which FIG. 7b view reveals the pathway within the catheter body for a coolant fluid used to inflate the catheter balloon.

### DESCRIPTION OF THE PREFERRED EMBODIMENTS

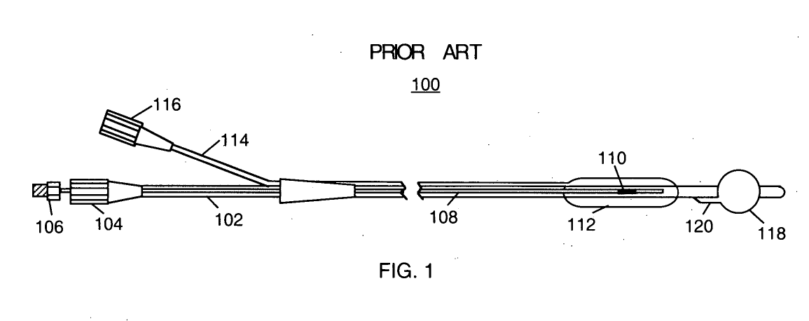

Referring to FIG. 1, there is shown an example of a typical prior-art microwave balloon catheter 100 that (1) employs an antenna internally situated within the interior of the balloon and (2) can be used in treating a male patient suffering from a disease of the prostate which results in an enlarged prostate that causes the bore of the urethra be narrowed. Microwave balloon catheter 100 comprises first lumen 102 terminated at its left end by first port 104. Microwave energy connector 106, attachable to first port 104, includes microwave coupling cable 108 extending through lumen 102 for forwarding microwave energy to microwave antenna 110. Surrounding microwave antenna 110 is balloon 112, which may be inflated by a fluid (i.e., a liquid or a gas) supplied thereto through second lumen 114 terminated at its left end by second port 116. Because catheter 100 is to be inserted into the urethra of a male patient for use in treating his enlarged prostate, it also includes conventional Foley balloon 118 which may be inflated by a fluid supplied thereto through third lumen 120 (which is only partially shown in order to maintain clarity of the more significant structure of the drawing).

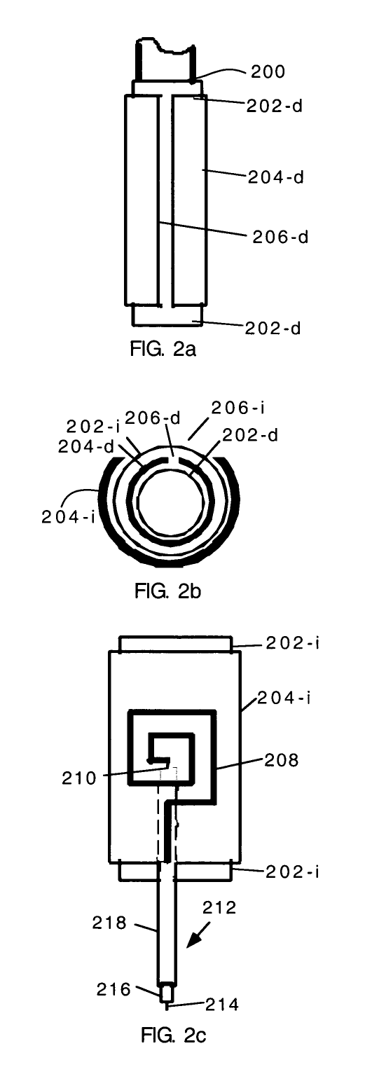

Referring now to the experimental embodiment of the present invention, shown in FIG. 2a is plastic catheter body 200, surrounded by deflated balloon 202-d, which, in turn, is surrounded by flexible cylindrical tubing 204-d that has a longitudinal split 206-d (where “d” represents the width of these elements in the deflated state of the balloon) situated on the back side of tubing 204-d. More specifically, elements 200 and 202-d consisted of a urethral catheter, manufactured by Celsion (Rockville, Md.) that is made of flexible plastic with a body diameter of approximately 7 mm and 48 mm length containing a water-pressure expandable balloon that is expandable to approximately 14 mm diameter when inflated. In its original unstretched state, element 204-d, consisted of a 45 mm approximate length of Masterflex 6424-18 silicon-rubber tubing, approximately 11 mm OD and 8 mm ID, (supplied by Cole-Parmer Instrument Co., Chicago, Ill.). Then, the lengthwise split 206-d opening was cut out of tubing 204-d.

As indicated in the end view shown in FIG. 2b (where “i” represents the width of these elements in the inflated state of the balloon), as long as balloon 202-d remains in its deflated state, the width of the split 206-d opening remains relatively narrow. However, when balloon 202-i is expanded to its inflated state 202-i, silicon-rubber tube 206 is stretched to accommodate inflated balloon 202-i. This results in the width of the split opening widening to 206-i. Specifically, first split tubing 204 is semi-flattened and placed in contact with the outer surface of deflated balloon 202-d. When semi-flattened tubing 204 is released, it tends to return to its original cylindrical form, so that, when placed over the slightly larger deflated balloon, it holds itself in place. In the inflated state of balloon 202i, the tubing 204 opens up to the extent the balloon diameter increases.

Shown in FIG. 2c is spiral antenna 208 (which is a directional antenna), attached to the front side of tubing 204, which surrounds inflated balloon 202-i. More particularly, tubing 204 was held flat and commercially available adhesive-backed copper tape (approximately 2 mm thick) was attached to completely cover the side of tubing 204 closest to the outer surface of inflated balloon 202-i to form a microstrip equivalent ground plane. A centrally-located small cutout hole 210 through the thickness of tubing 204 was provided. Then, on the other side of the flattened tubing 204, narrow strips of the same copper tape were cut and attached to form a microstrip line square spiral similar to that shown in FIG. 2c. The microstrip line started at the approximate center of the flattened tubing and ended with straight section that went to the center end of tubing 204, as shown in FIG. 2c.

A length of 0.085″ copper coaxial line 212 (such as Type KA50085 supplied by Precision Tube of Salisbury, Md.), which comprises center conductor 214, dielectric 216 and outer conductor 218, was inserted in between the outer surface of deflated balloon 202-d and the ground-plane side of tubing 204. Center conductor 214 of coaxial line 212 was placed through the small cutout hole 210 in the ground plane and soldered to the start of the microstrip spiral. The other end of the microstrip spiral was soldered to the outer conductor of coaxial line 212 using a small tab to bridge the thickness of tubing 204.

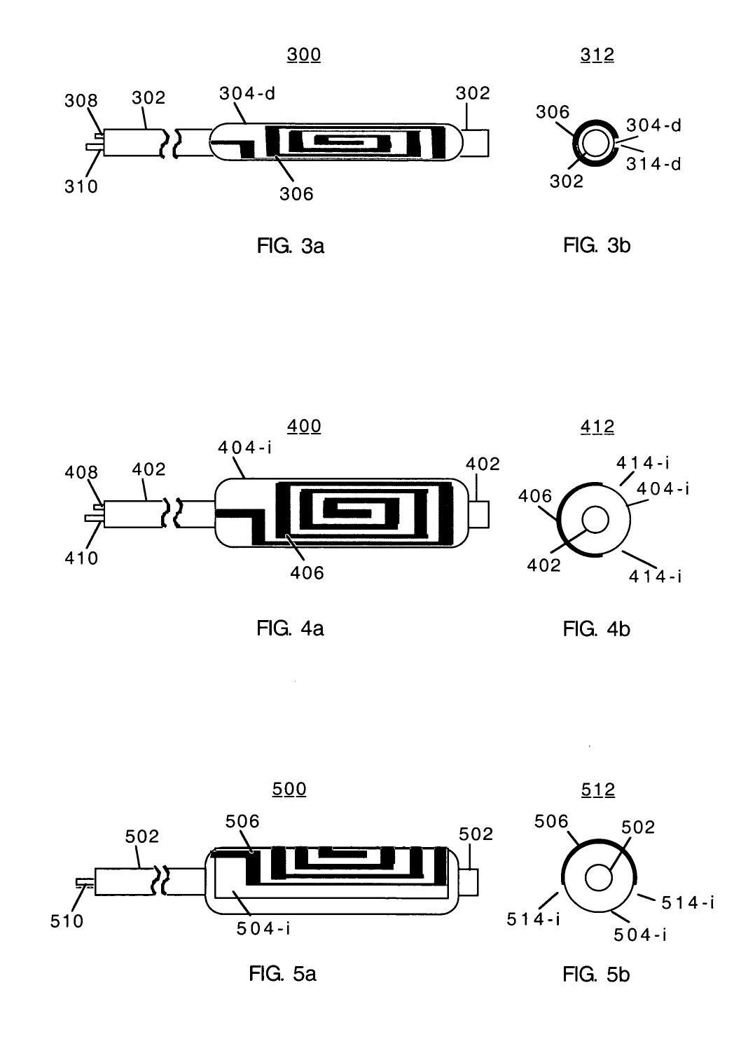

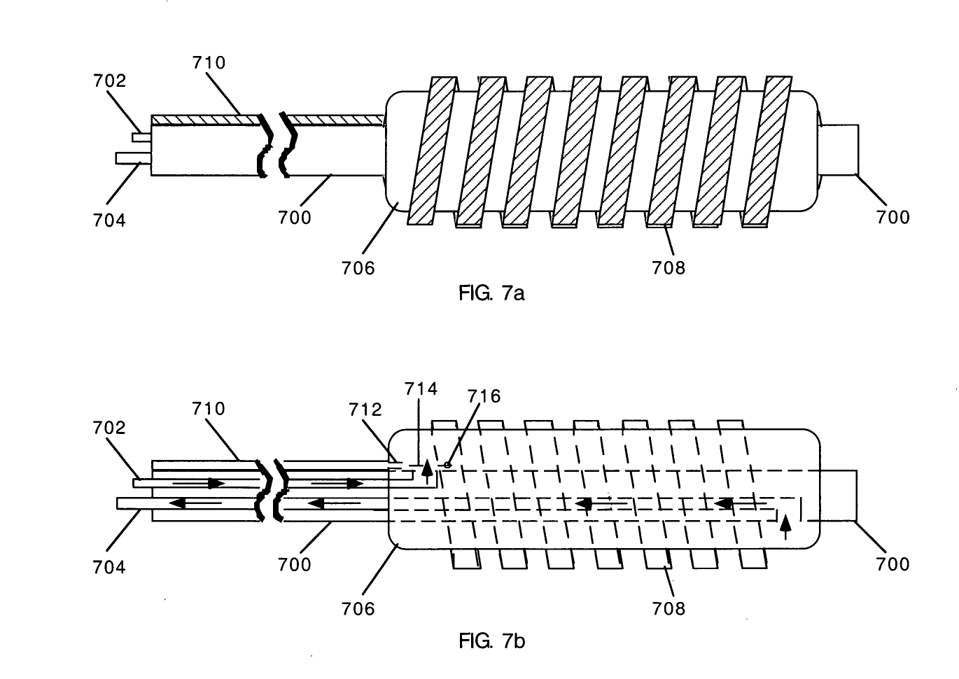

Referring to FIGS. 3a and 3b, there is shown a balloon-catheter that comprises a first preferred embodiment of the present invention, In particular, the longitudinal view 300 of this balloon catheter shown in FIG. 3a comprises catheter body 302 surrounded by balloon 304-d in a deflated state. As shown in, the longitudinal view 300, external directional spiral antenna structure 306 (1) faces front and (2) is situated in cooperative relationship with respect to the external surface of deflated balloon 304-d. Further, FIG. 3a shows the respective portions of inlet lumen 308 and outlet lumen 310 situated outside of catheter body 302. Inlet lumen 308 is used to transport a coolant fluid (either a gas or preferentially a liquid) which is used to fill and thereby inflate balloon 304 and outlet lumen 310 is used to extract coolant fluid from balloon 304. The inlet lumen 308 and outlet lumen 310, shown in their entirety along with the complete coolant pathway in the cutaway FIG. 7b drawing, are described in detail below. The end view 312 of this balloon catheter shown in FIG. 3b, which comprises catheter body 302 surrounded by balloon 304-d in a deflated state, also includes external directional spiral antenna structure 306 surrounding deflated balloon 304-d. External directional spiral antenna structure 306 (which is similar in structure to that of tubing 204 having spiral antenna 208 attached thereto) includes split 314-d. As shown in end view 312, split 314-d, which is situated on the back side of structure 306, appears on the right (so that the FIG. 3a front side of structure 306 appears on the left side in end view 312).

Referring to FIGS. 4a and 4b, there is shown views of the first preferred embodiment of the present invention which differs from the corresponding views thereof shown in above-described FIGS. 3a and 3b only in showing the balloon in an inflated state (rather than in the deflated state shown in FIGS. 3a and 3b). More particularly, in FIGS. 4a and 4b, elements 400, 402, 404-i 406, 408, 410, 412 and 414-i, correspond, respectively, with elements 300, 302, 304-d, 306, 308, 310, 312 and 313-d of FIGS. 3a and 3b.

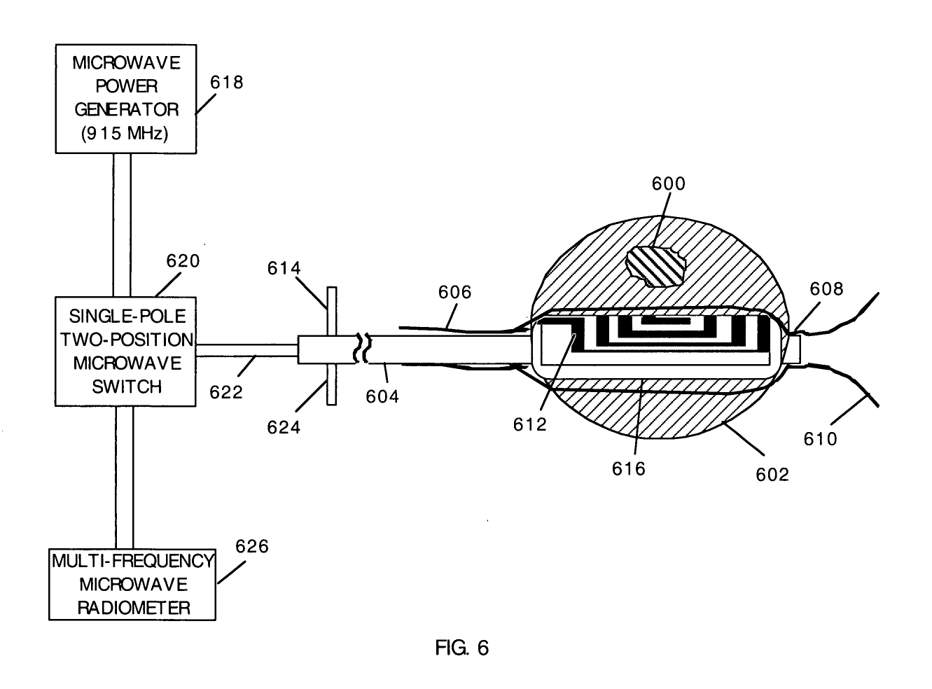

Referring to FIGS. 5a and 5b, there is shown views of the first preferred embodiment of the present invention which differs from the corresponding views thereof shown in above-described FIGS. 4a and 4b only in the entire FIGS. 4a and 4b structure has been rotated 90° about a longitudinal axis in the, FIGS. 5a and 5b views. Because of this 90° rotation, the input lumen is positioned in a vertical plane perpendicular to the paper directly below the output lumen and, therefore, does not appear in FIG. 5a. More particularly, in FIGS., 5a and 5b, elements 500, 502, 504, 506, 508, 510, 512 and 514-i, correspond, respectively, with elements 400, 402, 404-i, 406, 410, 412 and 414-i of FIGS. 4a and 4b. The orientation of the directional spiral antenna employed in the first preferred embodiment of the balloon catheter shown in views 500 and 512 is most suitable for use in the FIG. 6 system for treating a malignant tumor within a patient's diseased prostate tissue.

More specifically, FIG. 6 schematically shows malignant tumor tissue 600 situated within prostate tissue 602 (most often near the rectum) of a male patient. To treat tumor 600, (1) the first preferred embodiment of balloon catheter 604 in a deflated state is inserted in the patient's urethra 606, with its right end 608 in contact with the patient's bladder 610, (2); the balloon catheter is positioned so that its external directional antenna 612 is angularity oriented to radiate directly toward the location of tumor 600, and (3) then coolant fluid is supplied to inlet lumen 614 to effect the inflation of balloon 616 of catheter 604 to the view thereof shown in FIG. 6, wherein (a) the diameter of urethra 606 is expanded, thereby squeezing prostate tissue 602 and (b) maintaining external directional antenna 612 in intimate contact with urethral lining tissue overlying prostate 602 tissue. Power from microwave power generator 618 in the 915 MHz frequency band is supplied to external directional antenna 612 through a first position of single-pole, two position microwave switch 620 and coaxial feedline 622, resulting in microwave radiation transmitted from external directional antenna 612 and directed toward tumor tissue 600 effecting both the desired heating of the targeted malignant tumor tissue 600 and the undesired heating of the intervening healthy prostate tissue 602, as well as the lining tissue of urethra 606. Preferably, the frequency within the 915 MHz band should be varied until the best antenna match is determined by measuring the frequency at which the minimum amount of power is reflected and then operating at this optimum frequency. In order to prevent overheating of this intervening tissue (a maximum safe temperature is about 42° C.), the coolant fluid (which is preferentially a liquid, such as water, having a high heat capacity) is pumped through inlet lumen 614 to inflated balloon 616 and then continuously extracted from balloon 616 through outlet lumen 624. Further, single-pole, two position microwave switch 620 when in its second position (preferably switch 620 is continuously switched back and forth between its first and second positions) permits thermal radiation emitted by tumor tissue 600 and intervening tissue to be received by external directional antenna 612 (which is constructed to be sufficiently broadband to match transmitted radiation at a 915 MHz band microwave frequency and still match received radiation at thermal radiation microwave frequencies) and then forwarded over feedline 622 to multi-frequency microwave radiometer 626. This permits the temperature of these heated tissues to be continuously measured.

To minimize the amount of microwave power needed, it is desirable to maximize the proportion of the radiation absorbed by the targeted tumor tissue 600 and to minimize the proportion of the radiation absorbed by all of the intervening substance between the radiating antenna and the targeted tumor tissue 600. In the case of FIG. 6, where external directional antenna 612 is in direct contact with the lining tissue of urethra 606, the intervening substance is confined to only the lining tissue of urethra 606 and the healthy prostate tissue 602. This differs from the prior art, where the antenna is situated within the interior of the inflated balloon, so that the intervening substance also includes the coolant fluid. This would increase the amount of needed microwave power, which would cause undesirable heating of the coolant fluid (especially if the coolant is a high heat capacity liquid like water). Further, it would make it more difficult for the coolant fluid to remove sufficient heat from the lining tissue of urethra 606 to maintain it at a safe temperature no higher than 42° C. Further, eliminating losses in the cooling fluid results in cooler coaxial cables and, therefore, better radiometer accuracies. A more important factor in improving radiometer accuracy is that the use of an external antenna (rather than a prior-art internal antenna) avoids the coolant fluid (usually water) being situated between the tissue being heated and the external antenna. Because of the microwave lossiness of the water, the radiometer, in the prior-art antenna case, would be reading the water temperature more than the tissue temperature.

Although not shown in FIG. 6, the radiometric readings may be (1) fed back to microwave power generator 618 to control the power output thereof and (2) used to electronically vary the amount of cooling provide by the fluid coolant. This makes it possible to obtain optimum tissue temperature profiles in the prostate, (or, in general, in other tissues that are heated non-invasively with microwaves or radio frequencies). Also, thermocouples, infrared sensors or radiometers may be used to directly measure the urethral-lining surface temperature and maintain it at an optimum value.

When heating the prostate from only the urethra there are just 2 variables that the operator controls, i.e., the amount of cooling of the urethra and the amount of microwave power delivered to the urethra. However, 90% of all prostate cancers occur near the rectum. Therefore, in such cases, it would be desirable to employ an additional system similar to that shown in FIG. 6 with the balloon catheter thereof inserted in the rectum of the patient. In this case there would be 4 variables that the operator could control. Further, if two different microwave frequencies were used for the urethral system and the rectal system, there would be 6 variables. Based on readings of the surface and radiometric temperatures a computer could be used to control the amount of microwave heating and surface cooling in order to generate the desired optimum temperature distributions. In particular, the depth of heating is controlled by providing colder surface temperatures, which results in more power being delivered to the underlying diseased tissue (e.g., prostate malignant tumor tissue 600) without damaging the surface tissues. Thus, the deeper will be the depth of heating of the underlying diseased tissue.

When air cooling is used, one can electronically control the temperature of the cooling gas by controlling the amount of gas that escapes from an expansion valve. When water-cooling, is used, one can use mixtures of hot and cold water, and control the amount of each going into the mixture that cools the surfaces. Another option is Peltier cooling. Electronically controlled cooling would also be useful for treating other sites and diseases for example, recurrent breast cancer of the chest wall, psoriasis, etc.

Another benefit of employing an external antenna, such as external directional antenna 612, is that it produce better spatially defined heating patterns in the prostate than conventional water-cooled urethral microwave balloon catheters with antennas at their center. This is important because in conventional urethral balloon catheters the microwave fields that extend proximal from the balloon along the coaxial cables feeding the antennas tend to preferentially heat the sphincters because the tissues of the sphincters are closer to the cable while the tissues surrounding the prostatic urethra are further away because of the expansion balloons. As a result the amount of heating of the prostates with conventional microwave balloon catheters is limited by the requirement not to overheat the sphincters. With the disclosed balloon catheter, on the other hand, better “biological stents” (disclosed in the aforesaid prior-art U.S. Pat. No. 5,992,419) can be created in the urethra because the tissue surrounding the urethra can be safely raised to higher temperatures than is safely possible with conventional balloon catheters.

Th fact that external antenna 612 is highly directional is particularly useful when treating primary or recurrent prostate cancer, or when trying to prevent prostate cancer to occur in the future by non-invasively ablating prostate tissues in those parts of the prostate gland where malignancies are most likely to occur. To treat prostate cancer lesions the antenna would be aimed in the direction of the lesions. For example, to treat prostate cancer lesions near or in the direction of the rectum, external antenna 612 in the urethra would be aimed towards the rectum. As discussed above, external antenna 612 in the urethra, could work cooperatively with an additional external antenna in the rectum. In the treatment of prostate cancer, immunostimulants can be added the treatment, either systemically or by injecting into the treated region of the prostate. Thermally ablating prostate tissues also helps in the treatment of non-cancerous Benign Prostatic Hypertrophy (BPH) by reducing the pressure on the urethra. Note that the first treatments for BPH were done via the rectum. To treat BPH with a directional antenna, in the urethra, the catheter would be rotated during the treatment by deflating the catheter, rotating the catheter and re-inflating it. Also, in the treatment of BPH, the urethral external antenna could work cooperatively with an additional external antenna in the rectum.

The purpose of the system shown in FIG. 6 is to illustrate the use of a urethral balloon catheter incorporating the external antenna that forms the first preferred embodiment of the present invention (i.e., the case where the external antenna has a spiral configuration which renders it highly directional) to treat prostate disease. However, the present invention is neither limited to the treatment of prostate disease nor a balloon catheter employing an external highly-directional antenna having a spiral configuration. In this regard, reference is made to FIG. 7a, which shows a balloon catheter employing an external omnidirectional antenna having a helical configuration that forms a second preferred embodiment of the present invention. In particular, FIG. 7a comprises catheter body 700, coolant-fluid inlet lumen 702, coolant-fluid outlet lumen 704, inflated balloon 706, helical antenna 708 surrounding the external surface of inflated balloon 706 and coaxial feedline 710 for applying microwave power to helical antenna 708. Unlike a spiral microstrip antenna, does not require a ground plane.

The cutaway view of the second preferred embodiment of the balloon catheter shown in FIG. 7b shows that, at the distal end of coaxial feedline 710, dielectric 712 and inner conductor 714 thereof are exposed and the terminal end of inner conductor 714 is soldered at point 716 to the most proximate winding of helical antenna 708. Helical antenna 708 is effective as a monopole antenna that does not require connection to the outer conductor of coaxial feedline 710. This permits the structure of helical antenna 708 to comprise a spring which in its neutral state to have a relatively small diameter which is in proximity to balloon 706 in its deflated state. When balloon 706 is inflated, the spring tends to unwind under balloon pressure, thereby increasing its diameter so that it remains in proximity to balloon 706 in its inflated state. Thereafter, when balloon 706 is deflated, the restoring force of the spring returns it to its neutral state.

Further, the cutaway view of the second preferred embodiment of the balloon catheter shown in FIG. 7b indicates with arrows, pointing to the right, that the pathway for the coolant fluid entering balloon 706 extends through input lumen 702 and opening 710 in catheter body 700 into the proximate end of balloon 706 and indicates, with arrows, pointing to the left, that the pathway for the coolant fluid leaving balloon 706 extends from the distal end of balloon 706 through opening 710 in catheter body 700 and output lumen 704. In the case of each of FIGS. 3a, 4a, 5a and 6, the pathway for the coolant fluid flowing through the inlet lumen thereof and entering the balloon thereof and the pathway for the coolant fluid leaving the balloon thereof and flowing through the outlet lumen thereof is similar to the corresponding pathways of above-described FIG. 7b.

A balloon catheter incorporating an external antenna having a helical omnidirectional configuration would be particularly suitable for use as an interstitial probe, for treating sub-coetaneous diseased tissue of a patient, such as (1) deep-seated tumors and (2) varicose veins, as disclosed in the aforesaid prior-art U.S. patent application Ser. No. 10/337,159.

Although only (1) a first preferred embodiment of the present invention comprising a balloon catheter employing an antenna in cooperative relationship with an external balloon surface that has a spiral configuration and (2) a second preferred embodiment of the present invention comprising a balloon catheter employing an antenna in cooperative relationship with an external balloon surface that has a helical configuration have been specifically described herein, it is not intended that the present invention be limited to these two external-antenna configurations. Rather, the present invention is directed to any balloon catheter employing an antenna in cooperative relationship with an external balloon surface that is suitable for use in treating diseased tissue of a patient, regardless of the external antenna's particular configuration. Further, the structure of an antenna in cooperative relationship with an external balloon surface may be different from that specifically described above in FIGS. 2a, 2b and 2c. For instance the external antenna's configuration may comprise metallic printing directly of the external source of the balloon. (In the case of a spiral microstrip configuration, the metallic ground plane would be directly printed on the internal surface of the balloon.)

1. In a balloon catheter suitable for use in treating diseased tissue of a patient, wherein said balloon catheter comprises a catheter body, an inflatable balloon surrounding said catheter body, and an antenna, wherein in use (1) said catheter with said balloon in a deflated state may first be positioned so that said antenna is aligned with said patient's diseased tissue and (2) said balloon may then be inflated so that an exterior surface of said balloon presses said diseased tissue while said antenna transmits radiant energy to said diseased tissue thereby to effect the heating of said diseased tissue; the improvement wherein:
said antenna is longitudinally physically situated in cooperative relationship with said exterior surface of said balloon, thereby in use causing said inflated balloon pressing said diseased tissue to result in said antenna being in direct contact with irradiated tissue of said patient.

2. The balloon catheter defined in claim 1, wherein said catheter body comprises:
an input lumen that provides a first pathway for coolant fluid from a source situated outside of said balloon catheter to enter said balloon; and
an output lumen that provides a second pathway for said to leave said balloon and exit said balloon catheter.

3. The balloon catheter defined in claim 1, wherein:
said external antenna is a directional antenna.

4. The balloon catheter defined in claim 3, wherein:
said external directional antenna comprises a spiral microstrip structure.

5. The balloon catheter defined in claim 4, wherein said spiral microstrip structure comprises:
longitudinally-split plastic tubing having an inner longitudinal surface thereof enveloping said longitudinal external surface of said balloon with a metallic ground plane portion of said external directional antenna directly attached to said inner longitudinal surface of said tubing and a metallic spiral portion of said external directional antenna directly attached to an outer longitudinal surface of said tubing.

6. The balloon catheter defined in claim 1, wherein:
said external antenna is an omnidirectional antenna.

7. The balloon catheter defined in claim 6, wherein:
said external omnidirectional antenna comprises a metallic helical structure surrounding said longitudinal external surface of said balloon.

8. The balloon catheter defined in claim 1, wherein:
said external antenna is an external microwave antenna for transmitting microwave radiant energy to said diseased tissue while said balloon is inflated thereby to effect the heating of said diseased tissue.

9. In a system suitable for use in heat treating diseased prostate tissue of a patient, wherein said system comprises a balloon catheter including a catheter body, an inflatable balloon surrounding said catheter body, and an antenna; wherein in use (1) said catheter with said balloon in a deflated state may first be inserted into an orifice of said patient and positioned so that said antenna is aligned with said patient's prostate tissue and (2) said balloon may then be inflated so that an exterior surface of said balloon presses against lining tissue of said orifice that is adjacent to said patient's prostate tissue, the improvement wherein:
said antenna is a directional antenna that (1) is longitudinally physically situated in cooperative relationship with said exterior surface of said balloon, thereby in use causing said inflated balloon pressing against said lining tissue of said orifice that is adjacent to said patient's prostate tissue, to result in said antenna being in direct contact with said lining tissue of said patient and (2) transmits radiant energy of a given frequency band to said diseased prostate tissue in response to power within said given frequency band being supplied to said antenna; and
a power source and means including a feedline for supplying a given amount of power within said given frequency band to said external directional antenna, thereby to irradiate said diseased tissue and thereby effect the heating to a given therapeutic temperature.

10. The system defined in claim 9, wherein:
said given frequency band is the 915 MHz frequency band.

11. The system defined in claim 9, wherein said system further comprises a radiometer, and wherein:
said means including a feedline further includes a single-pole two-position switch for forwarding said given amount of power within said given frequency band from said power source to said feedline when said single-pole two-position switch is in a first switch position thereof and for forwarding thermal radiation received by said external directional antenna and supplied to said feedline to said radiometer when said single-pole two-position switch is in a second switch position thereof;
whereby said radiometer provides a reading indicative of the temperature of said irradiated diseased tissue.

12. The system defined in claim 11, wherein:
said means including a feedline further includes means for switching said single-pole two-position switch back and forth between its first and second switch positions thereby to continuously provide from said radiometer a reading of said irradiated diseased tissue's current temperature.

13. The system defined in claim 12, wherein said balloon catheter comprises:
means for supplying said balloon's interior volume with a coolant fluid for removing heat from said lining tissue of said orifice thereby to maintain the temperature of said lining tissue of said orifice at a safe temperature.

14. The system defined in claim 13, wherein:
said safe temperature is no higher than 42° C.

15. The system defined in claim 13, wherein said balloon catheter comprises a catheter body surrounded by said balloon thereof, and said means for supplying said balloon's interior volume with a coolant fluid comprises:
an input lumen in said catheter body that provides a first pathway for coolant fluid from a source situated outside of said balloon catheter to enter said balloon; and
an output lumen in said catheter body that provides a second pathway for said to leave said balloon and exit said balloon catheter.

16. The system defined in claim 15, wherein said orifice of said patient is said patient's urethra.

17. The system defined in claim 9, wherein said orifice of said patient is said patient's urethra.
